(19) United States
(12) Patent Application Publication
(10) Pub. No.: US 2009/0062765 A1
(43) Pub. Date: Mar. 5, 2009

(54) Discretion in absorbent garments

(76) Inventors: James R. Schermerhorn, Neenah, WI (US); Heather S. Mortell, La Crosse, WI (US)

Correspondence Address: KIMBERLY-CLARK WORLDWIDE, INC.;Catherine E. Wolf, 401 NORTH LAKE STREET, NEENAH, WI, 54956, US

(21) Appl. No.: 11/897,907

(22) Filed: Aug. 31, 2007

(51) Int. Cl. A61F 13/15

(52) U.S. Cl. 604/393; 604/358

(57) ABSTRACT

A hygiene discretion system includes at least two articles where at least one is an absorbent garment including a shell with an absorbent member attached to the shell and at least one non-absorbent garment. The shell of the disposable absorbent garment substantially matches the non-absorbent garment. In some aspects, a secondary garment is also provided which coordinates with one of the other garments. In some aspects, replacement garments are provided. Any number of the garments may be placed in the same container.

### BACKGROUND

Articles, such as absorbent articles, are useful for absorbing many types of fluids, including fluids secreted or eliminated by the human body. For some persons it is necessary to wear disposable absorbent garments about the lower torso to absorb and contain bodily exudates. For instance, some persons have, or develop, physical or cognitive problems where they cannot adequately control the bowel or bladder and are considered incontinent.

This may happen all of the time, or under certain situations, for example during sleep, exercise, stress, etc. This can include a health condition termed enuresis where young persons who are otherwise toilet-trained do not wake at night when their bladder needs to be voided. These persons can accidentally wet their beds until they become physically and/or mentally mature enough to prevent the bedwetting incidents.

For all such persons, regardless of age, incontinence can prove to be at times embarrassing, and lead to distress in social situations where their problem may be discovered by others. In an effort to assist those persons suffering from incontinence, disposable absorbent garments have been developed that effectively contain bodily exudates. However, such protective undergarments may look like diapers or training pants used by babies and small children. For some persons (such as older children), this causes a loss of dignity, and may lead to the avoidance of any social situation where others may accidentally see them in their protective undergarment. For others, it may lead to an aversion to using protective undergarments. For example, many such persons may be reluctant to participate in sleepovers or retreats, or to change clothes in locker rooms, because they do not want others to discover their condition. Therefore, there is a need for a system or method which allows a user to utilize an absorbent article while maintaining discretion as to the user's need for the article.

### SUMMARY

In response to the needs discussed above, a hygiene discretion system is provided comprising a disposable absorbent garment and a non-absorbent garment. The disposable absorbent garment comprises a shell and an absorbent member. Desirably, the shell substantially matches the non-absorbent garment. In some aspects, the disposable absorbent garment and non-absorbent garment are each a boxer.

In some aspects, the shell substantially matches the non-absorbent garment via a garment theme. In other aspects, the garment theme comprises a style. In still other aspects, the garment theme comprises a color. In yet other aspects, the garment comprises a fabric texture. In still other aspects, the garment theme comprises a print pattern. In yet other aspects, the garment theme is selected from style, color, fabric pattern, print pattern, or combinations thereof.

In some aspects, the shell has an opacity of at least 50%, as measured by the ISO Opacity Test. In other aspects, the disposable absorbent garment and the non-absorbent garment comprise substantially the same looseness, as measured by the Garment Looseness Test. In still other aspects, the non-absorbent garment and the shell each cover at least 10% more surface area than the absorbent member of the disposable absorbent garment, as measured by the Surface Coverage Test.

In some aspects, the hygiene discretion system further comprises a container. In other aspects, the disposable absorbent garment and the non-absorbent garment are provided in the same container.

In some aspects, the hygiene discretion system comprises a secondary garment. In other aspects, the secondary garment is a shirt. In still other aspects, the secondary garment coordinates with the non-absorbent garment. In yet other aspects, the hygiene discretion system further comprises a container where the disposable absorbent garment, the non-absorbent garment, and the secondary garment are provided in the same container.

In some aspects, the hygiene discretion system further comprises at least one replacement garment, wherein the at least one replacement garment substantially matches the non-absorbent garment. In other aspects, the at least one replacement garment is provide in a separate container from the disposable absorbent garment and non-absorbent garment.

In some aspects, a method of providing a system for enabling discretion comprises: providing a disposable absorbent garment; providing a non-absorbent garment; providing a container; bundling the disposable absorbent garment and the non-absorbent garment to form a bundled discretion product offering; and providing the bundled discretion product offering to a user; wherein the disposable absorbent garment comprises a shell and an absorbent member; wherein the shell coordinates with the non-absorbent garment; and wherein the disposable absorbent garment and the non-absorbent garment are each a boxer. In other aspects, the method further comprises the step of providing a replacement garment, which optionally may be bundled with the disposable absorbent garment and/or the non-absorbent garment. In yet another aspect, the method further comprises the step of providing at least one replacement garment, which optionally may be bundled with the disposable absorbent garment and/or the non-absorbent garment.

Numerous other features and advantages of the present invention will appear from the following description. In the description, reference is made to exemplary embodiments of the invention. Such embodiments do not represent the full scope of the invention. Reference should therefore be made to the claims herein for interpreting the full scope of the invention. In the interest of brevity and conciseness, any ranges of values set forth in this specification contemplate all values within the range and are to be construed as support for claims reciting any sub-ranges having endpoints which are real number values within the specified range in question. By way of a hypothetical illustrative example, a disclosure in this specification of a range of from 1 to 5 shall be considered to support claims to any of the following ranges: 1-5; 1-4; 1-3; 1-2; 2-5; 2-4; 2-3; 3-5; 3-4; and 4-5.

### FIGURES

The foregoing and other features, aspects and advantages of the present invention will become better understood with regard to the following description, appended claims and accompanying drawings where:

FIG. 1 is a rear elevational view a disposable absorbent garment of the present invention and a substantially matching non-absorbent garment;

FIG. 2 is a front elevational view of a disposable absorbent garment of the present invention where the shell has a boxer configuration in the form of a skort;

FIG. 3 is a front perspective view of a disposable absorbent garment of the present invention;

FIG. 4 is a side perspective view of the garment shown in FIG. 3, with a side opened for easy view of the garment interior;

FIG. 5 is a plan view of an absorbent member utilized in the disposable absorbent garment of FIG. 3;

FIG. 6 is an aspect of the invention comprising a disposable absorbent garment, a non-absorbent garment and a secondary garment;

FIG. 7 is an aspect of the invention comprising a disposable absorbent garment, a non-absorbent garment and a replacement garment; and

FIG. 8 is a disposable absorbent garment and a non-absorbent garment, both of which substantially match based on a boxer short style having a print pattern theme.

Repeated use of reference characters in the present specification and drawings is intended to represent the same or analogous features or elements of the present invention.

### Test Methods

### ISO Opacity Test

The Opacity of the test materials may be tested using a Technibrite Micro TB-1C apparatus, available from Technidyne Corporation, New Albany, Ind., U.S.A.

Test material samples consist of garment portions such as a portion of shell from a disposable absorbent garment of the present invention. The samples should be clean and free of defects, and the testing areas on the samples to be analyzed should be free from perforation lines and seams. If the shell is not of a solid color, then the garment must be divided into as many test material specimens as possible. An attempt must be made to include all indicia or color value gradations disposed on the outer surface if the garment.

The samples are cut into square shapes approximately 2.25 inches (5.7 cm) on each side. If the specimens are of a solid color, at least 10 specimens should be tested individually. If the specimens are not of a solid color, then at least 50 specimens should be tested individually. Stacks of specimens should be used as “full opacity” controls; the number of specimens per stack should be chosen so that no noticeable change in readings results when additional sheets are added. Each stack should be used as a control for each of five different individual specimens; therefore, for 10 individual specimens, two “full opacity” control stacks will be required.

The tests should be conducted in a standard laboratory atmosphere of 23±1° C. (73.4±1.8° F.) and 50±2% relative humidity. A black body cup must be on the sample holder for all single-sheet opacity readings. A Y (green) filter should be in the active position. The infinite stacks and individual specimens should remain in order; i.e., stack 1 with specimens 1-5; stack 2 with specimens 6-10; etc.

The instrument is turned on and allowed to warm up for at least 30 minutes before testing any samples. The instrument is calibrated as directed in the instrument manufacturer's instruction manual. The calibration is verified at least once per day, prior to testing. The swing-in standard should be kept clean and free of fingerprints, using lint-free wipes or lens paper and a lens cleaner that does not leave a residue, as needed.

Data obtained are expressed as percent opacity, where the “full opacity” control stack represents 100%. The opacity value for a material should be calculated by averaging the percent opacity values for at least 10 individual specimens of that material if of a solid color, or at least 50 individual specimens if not of a solid color.

The following procedure should be used:
1. Press the REPROGRAM button and select ISO OPACITY for data output.
2. Press PRINT to complete the routine and check the selected output type for correctness.
3. Press the QC button, enter the specimen number, and press PRINT again.
4. Load stack 1 onto the spring-loaded sample holder. Release the spring-loaded sample holder, with the stack, so it is tight against the port.
5. Press SCAN.
6. Remove the stack.
7. Place individual specimen 1 onto the spring-loaded sample holder. Be sure that the specimen is flat and tight against the port. Press PRINT.
8. Repeat steps 4-7 with successive specimens until individual specimens 1-5 have each been tested.
9. Repeat steps 2-8 for stack 2 and specimens 6-10; these steps can be repeated as many times as needed until all individual specimens have been tested against their respective stacks. Keep individual samples and sets in order.
10. Average the individual “Calculated TAPPI opacity” values from the full set of tests.

### Garment Looseness Test

The garments of the present invention should be analyzed for looseness as follows: The garment should be removed from the package and gently spread flat with front side up (i.e., with legs or leg openings side by side, rather than overlapping), without stretching. The garment should be marked at a location 6 inches (15 cm) below the top waist edge of garment, at least 10 locations approximately evenly spaced (such as 5 on the garment front, 5 on the garment back). The width of the flattened garment should be measured along the line of marking points, and multiplied by two. This number serves as the effective hip circumference of the garment.

If the garment contains a shell, the shell only is marked as described. If the shell is gathered, elasticized or otherwise non-flat in the relaxed garment, such as may result from an elastic waist region, the shell is separated from the elasticized waistband or other gathered area after the shell is marked, such as by cutting. Separation is done without stretching or otherwise deforming the material of the shell. The shell should then be gently spread flat and extended as described above to provide a generally linear array of marking points. The width of the flattened shell is measured along the line of marking points, and multiplied by two. This number serves as the effective hip circumference of the garment.

The effective hip circumference of the garment is compared to the mean hip circumference of a wearer at the mid-point of the garment's intended weight range (male and female combined, for garments intended for use by either gender, or that of a specific gender when the garment is intended for use by only one gender). Anthropometric data such as that seen in Table 1 may be used for a given age range. It is desirable to update the anthropometric data as such data tend to change over time. A garment with effective hip circumference equal to or less than the mean hip circumference of wearers at the mid-point of the garment's intended weight range is classified as a close-fitting garment. A garment with effective hip circumference greater than the mean hip circumference of wearers at the mid-point of the garment's intended weight range is classified as a loose-fitting garment. A first garment that has an effective hip circumference that is larger than a second garment effective hip circumference is more loose-fitting than the second garment.

Data from an anthropometric study of hip circumference and weight is shown in Tables 1 and 2, respectively. (Table data source: Sandy Ressler, AnthroKids—Anthropometric Data of Children, United States National Institute of Standard and Technology, 1977). The hip circumference is defined as the maximum horizontal circumference of the hips at the level of the greatest posterior protrusion of the buttocks, as viewed from the side of an individual standing erect.

TABLE 1
HIP CIRCUMFERENCE CM. - (MALES AND FEMALES)

| AGE/YRS | N | MEAN | S.D. | MIN | 5TH | 50TH | 95TH | MAX |
|---|---|---|---|---|---|---|---|---|
| 2.0-3.5 | 200 | 51.7 | 3.2 | 43.8 | 46.9 | 51.8 | 57.0 | 62.1 |
| 3.5-4.5 | 225 | 53.5 | 3.5 | 45.4 | 48.2 | 53.1 | 59.4 | 65.6 |
| 4.5-5.5 | 260 | 56.1 | 3.5 | 48.2 | 51.2 | 55.6 | 62.2 | 77.7 |
| 5.5-6.5 | 217 | 58.3 | 4.0 | 49.8 | 52.0 | 58.0 | 65.7 | 70.0 |
| 6.5-7.5 | 222 | 61.9 | 5.2 | 49.1 | 55.2 | 60.9 | 71.7 | 84.6 |
| 7.5-8.5 | 188 | 65.0 | 5.6 | 55.9 | 57.7 | 64.0 | 76.2 | 90.8 |
| 8.5-9.5 | 248 | 67.7 | 5.9 | 55.3 | 59.4 | 67.0 | 79.8 | 91.4 |
| 9.5-10.5 | 247 | 70.8 | 6.7 | 59.4 | 61.7 | 69.8 | 82.8 | 108.5 |
| 10.5-11.5 | 276 | 74.3 | 7.7 | 59.3 | 64.2 | 73.1 | 90.6 | 108.2 |
| 11.5-12.5 | 283 | 76.6 | 7.2 | 62.3 | 66.2 | 75.7 | 90.1 | 101.9 |
| 12.5-13.5 | 310 | 81.0 | 7.3 | 64.8 | 70.0 | 79.8 | 93.3 | 107.3 |
| 13.5-14.5 | 270 | 84.3 | 7.1 | 60.2 | 72.9 | 84.3 | 96.5 | 104.2 |
| 14.5-15.5 | 262 | 88.1 | 7.1 | 69.5 | 75.6 | 88.2 | 99.0 | 115.9 |
| 15.5-16.5 | 198 | 90.5 | 6.3 | 67.1 | 80.9 | 90.1 | 101.5 | 108.9 |
| 16.5-17.5 | 220 | 91.8 | 6.4 | 78.7 | 82.9 | 91.0 | 104.6 | 116.0 |
| 17.5-19.0 | 156 | 93.0 | 6.0 | 77.1 | 84.2 | 92.4 | 103.4 | 112.5 |

TABLE 2
WEIGHT KG. - (MALES AND FEMALES)

| AGE/YRS | N | MEAN | S.D. | MIN | 5TH | 50TH | 95TH | MAX |
|---|---|---|---|---|---|---|---|---|
| 2.0-3.5 | 212 | 14.1 | 1.9 | 10.1 | 11.3 | 14.0 | 17.1 | 20.7 |
| 3.5-4.5 | 228 | 16.2 | 2.1 | 11.4 | 13.1 | 16.0 | 19.6 | 23.8 |
| 4.5-5.5 | 271 | 18.3 | 2.5 | 13.7 | 15.1 | 17.9 | 22.1 | 36.0 |
| 5.5-6.5 | 243 | 20.5 | 3.0 | 13.1 | 16.1 | 20.0 | 25.6 | 32.5 |
| 6.5-7.5 | 231 | 23.7 | 4.1 | 15.4 | 18.3 | 22.9 | 31.1 | 43.6 |
| 7.5-8.5 | 197 | 26.6 | 5.2 | 17.6 | 20.4 | 25.5 | 36.9 | 54.2 |
| 8.5-9.5 | 256 | 29.7 | 5.5 | 19.6 | 22.7 | 28.6 | 40.0 | 60.0 |
| 9.5-10.5 | 258 | 33.1 | 6.6 | 22.2 | 24.8 | 31.9 | 45.5 | 69.0 |
| 10.5-11.5 | 282 | 37.2 | 8.2 | 20.7 | 27.4 | 35.2 | 53.2 | 80.0 |
| 11.5-12.5 | 287 | 40.3 | 8.1 | 26.1 | 29.3 | 39.1 | 54.7 | 77.8 |
| 12.5-13.5 | 315 | 45.7 | 9.0 | 26.5 | 33.6 | 44.2 | 62.0 | 85.8 |
| 13.5-14.5 | 271 | 50.8 | 9.2 | 29.8 | 37.7 | 50.0 | 67.8 | 77.3 |
| 14.5-15.5 | 264 | 55.5 | 10.0 | 32.7 | 40.2 | 54.2 | 72.1 | 105.0 |
| 15.5-16.5 | 198 | 60.7 | 11.5 | 33.0 | 44.7 | 58.9 | 80.1 | 106.6 |
| 16.5-17.5 | 221 | 62.2 | 11.6 | 36.4 | 47.0 | 60.1 | 81.1 | 106.2 |
| 17.5-19.0 | 156 | 65.7 | 13.1 | 41.4 | 45.6 | 65.4 | 86.5 | 112.3 |

### Surface Coverage Test

The extent of coverage of body surface area provided by two different garments may be assessed visually, such as by examination of photographs of a mannequin wearing both garments. One such approach involves taking photographs of a mannequin wearing each garment. Photographs are taken from the same perspective (i.e. relative vertical placement), at the same distance from the mannequin, and the mannequin is disposed in two positions relative to the camera (front facing and rear facing). For each garment, there is one picture taken from the front and one from the back with the mannequin unclothed, and several other pictures taken with the mannequin wearing a garment specimen. A sample of four identical specimens having identical sizes are used for a total of 10 photographs (four front and four rear views with garments, one front and one rear view without garments). The pictures are analyzed by determining the mean surface area of the mannequin covered by the garment after combining results from both the front and the rear views. This can be accomplished by printing the pictures and cutting out the area unclothed and the area clothed, and weighing the pieces of paper. The clothed weights and the unclothed weights are then averaged. The averages are then used to form a ratio of coverage. Alternatively, computer software may be used which can accurately analyze the pictures and provide the mean surface area of the mannequin covered by the garment.

### DEFINITIONS

It should be noted that, when employed in the present disclosure, the terms “comprises,” “comprising” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

When introducing elements of the present invention or the preferred embodiment(s) thereof, the articles “a”, “an”, “the” and “said” are intended to mean that there are one or more of the elements.

“Disposed,” “disposed on,” “disposed with,” “disposed at,” “disposed near” and variations thereof are intended to mean that one element can be integral with another element, or that one element can be a separate structure bonded to or placed with or placed near another element.

The term “absorbent” when used to describe an article, such as a personal care article, generally refers to devices which can absorb and contain fluids. For example, personal care absorbent articles refer to devices which are placed against or near the skin to absorb and contain the various fluids discharged from the body.

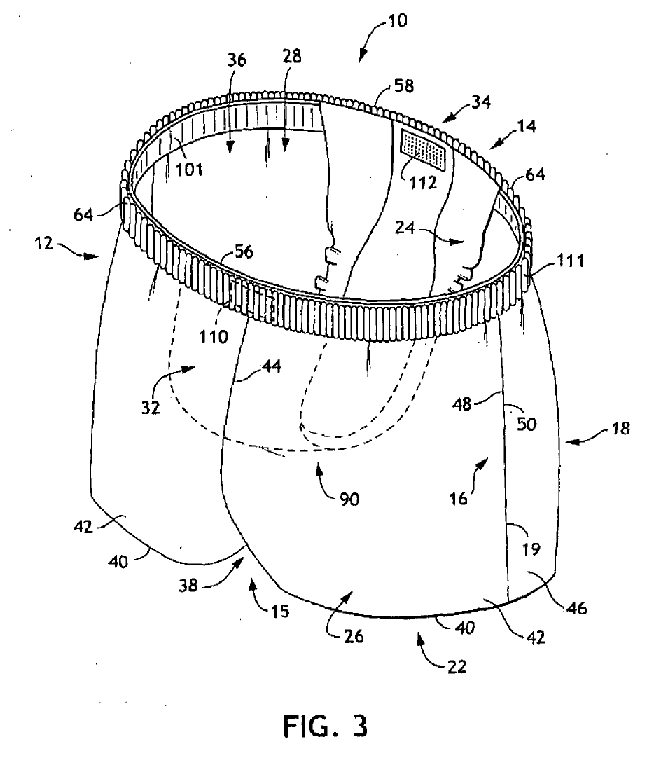

The term “boxer” is used to describe a garment that is intended to be worn around the lower torso. As used herein, the term “boxer” includes boxer shorts (see FIG. 3), skorts 900 (See FIG. 2), trousers (a boxer short with longer leg portions—not shown), bloomers (a boxer short with leg portions that are gathered at or near the lower edges to hug the leg or thigh of the user—not shown), briefs (substantially form fitting with short or no leg portions—not shown), or boxer briefs (substantially form fitting with leg portions—not shown). In the case of disposable absorbent garment in the form of a boxer, the boxer has a shell attached to an absorbent member. This shell can range between being loose fitting to form fitting (close fitting) onto the absorbent member or onto the user, and may be constructed from a stretch material or other non-absorbent material. The shell may be attached directly to the absorbent member either fully or partially, such as at the waist. See U.S. Publication No. 2005/0256473, incorporated herein by reference in a manner that is consistent herewith.

The term “container” refers to the device which contains the garments of the present invention for storage, viewing and/or purchasing by a consumer, such as individual product packaging, in a condition that is typically placed on a store shelf.

The term “disposable” is used herein to describe absorbent articles that are not intended to be laundered or otherwise restored or reused as an absorbent article after a single use.

The term “life stage” is used herein to refer to where a user of the present invention is at maturity-wise in his or her life. The users of the garments of the present invention may wear any appropriately-sized garments of the system and method. Of course, users may eventually determine that there is no need to wear any of the garments of the present invention if the reason for which the garments are used ceases to exist; for example, the user no longer experiences incontinence.

The term “non-absorbent” when used to describe a garment of the present invention refers to a garment which does not include a separate absorbent member, as described herein. For example, a boxer comprising a nonwoven shell and an absorbent member attached thereto would be considered an absorbent garment, while a boxer comprising the same nonwoven shell, but without the absorbent member attached thereto would be considered a non-absorbent garment. Likewise, for example, a woven boxer without a separate absorbent member attached thereto would also be considered a non-absorbent garment for purposes of the invention.

The term “substantially matches” as used herein means that when the disposable absorbent garment and the non-absorbent garment of the present invention are observed separately (rather than side-by-side) by an adult having 20/20 vision from a distance of at least one (1) meter in an environment providing 30 footcandles (320 Lux) of illumination, the garments are perceived as being the same garment by the average consumer. Thus, slight variations that could be detected within one (1) meter, or when side-by-side, would be acceptable and still considered to be substantially matching. Such variations could include slight variations in: style (e.g., a looser fit or longer legs for one of the garments); print pattern (e.g., print design or the percentage of area covered by each color in a print repeat); and color hue or other measures of color (e.g., Hunter color index or CIE-LAB color space).

The term “superabsorbent” refers to water-swellable, water-insoluble organic or inorganic materials capable, under the most favorable conditions, of absorbing at least about 5 times their weight, or at least about 10 times their weight, or at least about 20 times their weight in an aqueous solution containing 0.9 weight percent sodium chloride.

The term “woven” refers to garments in the category of textiles, such as those made out of cloth or other textile fabrics.

These terms may be defined with additional language in the remaining portions of the specification.

### DETAILED DESCRIPTION

A hygiene discretion system, and method of providing a system for enabling discretion, of the present invention includes at least two articles. More particularly, the invention includes at least one disposable absorbent garment and at least one non-absorbent garment. The disposable absorbent garment comprises a shell with an absorbent member at least partially attached to the shell, such as within the circumference of the shell for example. In addition, the shell of the disposable absorbent garment substantially matches the external look of the non-absorbent garment. Thus a user can discreetly alternate between the disposable absorbent garment and the non-absorbent garment without others taking notice.

Each garment may offer a different type of discretion for the user; that is, the non-absorbent garment may be desirably worn during times of continence of the user, while the disposable absorbent garment is desirably worn during potential times of incontinence of the user. In some aspects, garments are desirably designed for use by a person at a single life stage and may be particularly suitable for situation where at least portions of the garment may be seen by others. In some aspects, each garment may be manufactured by or for the same business entity and target the same user group.

Generally, a user of the present invention experiences difficulty controlling his or her bowel or bladder habits, either all of the time, or at certain times of day, or in certain situations. Since such users suffer from incontinence, which may result from medical conditions such as hormonal imbalance, stroke, senility, cognitive delays, brain damage, and paralysis, or may be experienced during normal growth and development such as the process of toilet training in children, it is anticipated that the user of the present invention may be as young as about 2 years old; however, there is no limit as to how old the user might be.

The typical user of the present invention will typically have a desire or need to conceal his or her incontinence. Some users may want to have a more discreet garment for traveling outside the home, but may also desire a discreet garment for use in the privacy of his or her home as well. For example, a young person that experiences enuresis may want to participate in social situations such as sleeping away from home, for example at a friend's or relative's home. In another example, a person may wish to attend a retreat or go camping with a group of peers. In still another example, a person may wish to change clothes in a locker room prior to, or after, exercising or playing a sport. For these and other such circumstances, the user may desire a garment that remains discreet, especially by the way the garment looks when worn by a user. More particularly, the user may desire a disposable absorbent garment, such as a boxer in this aspect, which substantially matches a similar non-absorbent garment. Thus, in times when continence is anticipated, the user may choose to wear a non-absorbent garment, such as a woven or nonwoven boxer. However, in times when absorbency may be needed, the user can discreetly replace the non-absorbent boxer with a substantially matching disposable absorbent boxer, without other persons noticing a difference. Such discretion can greatly improve the quality of life for persons experiencing incontinence.

For ease of explanation, the description hereafter will be in terms of absorbent products directed to users suffering from enuresis-type incontinence. However, this is not meant to limit the invention to products directed toward this type of incontinence. It is understood that absorbent products of the present invention also include, but are not limited to, those for adult incontinence and menstruating women, among others.

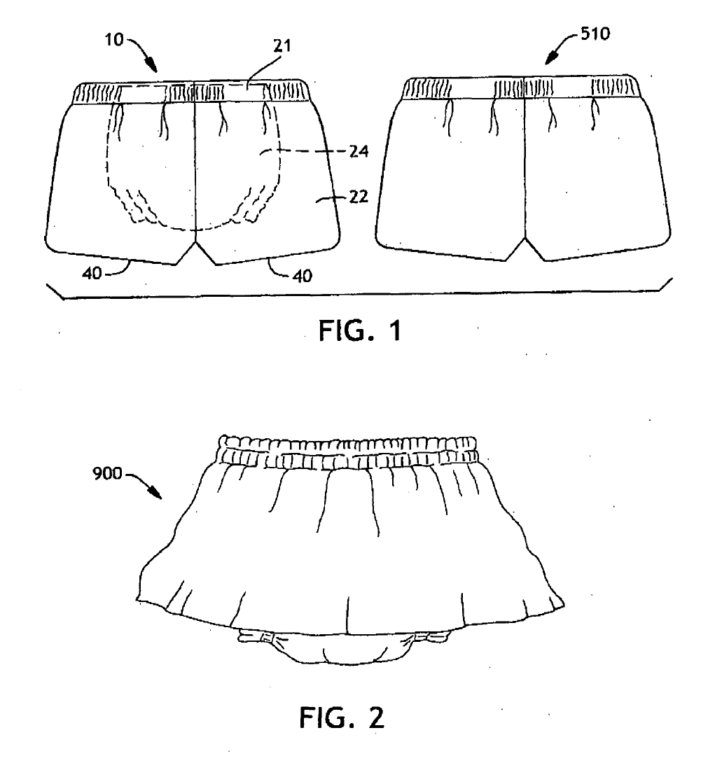

To gain a better understanding of the present invention, attention is directed to FIG. 1 which, for exemplary purposes, shows a hygiene discretion system and method of the present invention. Provided is a disposable absorbent garment 10 and a non-absorbent garment 510. In this particular aspect, the disposable absorbent garment 10 and the non-absorbent garment 510 are each a boxer, more particularly a boxer short style. As used herein, the term “style” refers to the type of garment (i.e., boxer short, boxer brief, bloomer, etc.). The disposable absorbent garment 10 includes a shell 22 with a waist band area 21 and leg portions 40, and a separate absorbent member 24 attached thereto. In this aspect, the absorbent member 24 is partially attached to the shell 22 at the waist band area 21 and is located within the shell's circumference.

In desirable aspects of the present invention, the disposable absorbent garment 10 may be bundled with at least one non-absorbent garment 510 of the same style such that the disposable absorbent garment 10, and in particular the shell 22 of the disposable absorbent garment 10, substantially matches the external look of the non-absorbent garment 510. This provides the opportunity for the user to switch from a non-absorbent garment to a disposable absorbent garment discreetly, since the disposable absorbent garment substantially matches the product worn by the user immediately prior to the switch.

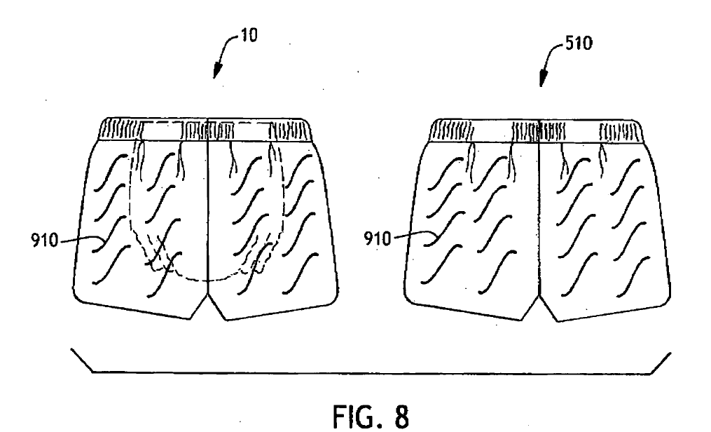

In some aspects, to ensure a closer match, it may be desirable that the disposable absorbent garment 10 and the non-absorbent garment 510 substantially match each other by including a garment theme. For example, referring to FIG. 8, garment themes can include, but are not limited to, garment color (not shown), garment fabric texture (not shown), garment print pattern 910 (FIG. 8), and combinations thereof.

Certain physical properties of the garments may also be desirable to ensure a closer match between the shell 22 of the disposable absorbent garment 10 and the non-absorbent garment 510. Such properties can be measurable and include, but are not limited to, garment opacity, garment looseness and garment coverage area, for example.

Garment opacity is determined using the ISO Opacity Test described above. With respect to opacity of a garment shell 22, it is desirable that the opacity be at least 50 percent in some aspects of the invention. In other aspects of the invention, it is desirable that the opacity be at least 60 percent. In still other aspects of the invention, it is desirable that the opacity be at least 70 percent. In yet other aspects of the invention, it is desirable that the opacity be at least 80 percent. The higher the opacity of shell 22, the more discreet the garment 10 is because there is less ability for others to see the absorbent member through the shell when the garment 10 is worn.

Garment coverage area is determined using the Surface Coverage Test. With respect to the coverage area for any garment in the system and method of the present invention, such as garment 10 or 510, it is desirable that the shell 22 covers a larger surface area of the wearer than the absorbent member 24 attached thereto. Each garment of the present invention may be configured to fit a wearer in a particular size range, or having about the same mean hip circumference. In one aspect, both the non-absorbent garment 510 and the shell 22 of the disposable absorbent garment 10 may cover at least 10% more surface area than the absorbent member 24 of the disposable absorbent garment 10. In another aspect, the non-absorbent garment 510 and the shell 22 may cover at least 15% more surface area than the absorbent member 24. In still another aspect, the non-absorbent garment 510 and the shell 22 may cover at least 20% more surface area than the absorbent member 24. In yet another aspect, the non-absorbent garment 510 and the shell 22 may cover at least 25% more surface area than the absorbent member 24.

Garment looseness is determined using the Garment Looseness Test described above. A “loose fit” will require that the effective hip measurement of a garment be larger than the mean hip measurement of a wearer of a predetermined weight. The corresponding hip size and weight can be determined by the age ranges listed in Tables 1 and 2 above. For example, a person having a mean weight of 45.7 kg has a mean hip circumference of 81 cm. For example, in some aspects, it may be desirable that the disposable absorbent garment 10 and the non-absorbent garment 510 comprise substantially the same looseness. In other aspects, it may be desirable that one has a looser fit than the other, provided that the garments substantially match, as defined herein.

In some aspects, the disposable absorbent garment 10 of the present invention can have a relatively loose fitting shell. The term “shell” refers to an external member of the disposable absorbent garment to which may be partially attached an absorbent member, typically at the waist region. The term “absorbent member” includes diapers, training pants, and the like. Several patents and patent applications disclosing shell-covered garments, and are incorporated herein by reference in a manner that is consistent herewith: US2005/0131377 to Franke et al.; U.S. Pat. No. 6,168,585 to Cesco-Cancian; U.S. Pat. Nos. 6,115,847 and 6,009,558 and 5,876,394 to Rosch et al.; U.S. Pat. Nos. 6,192,521 and 5,915,536 to Alberts et al.; EP 1329166 and EP 0955975 to Rosch et al.; U.S. Ser. No. 10/737101 (US20050131382); U.S. Ser. No. 10/735978 (US20050131377); U.S. Ser. No. 10/736069 (US20050131381); U.S. Ser. No. 10/736443 (US20050125879). Each of these references is commonly owned by the assignee of the present invention. Because the shell may be relatively loose, the absorbent member underneath the shell may not be readily apparent.

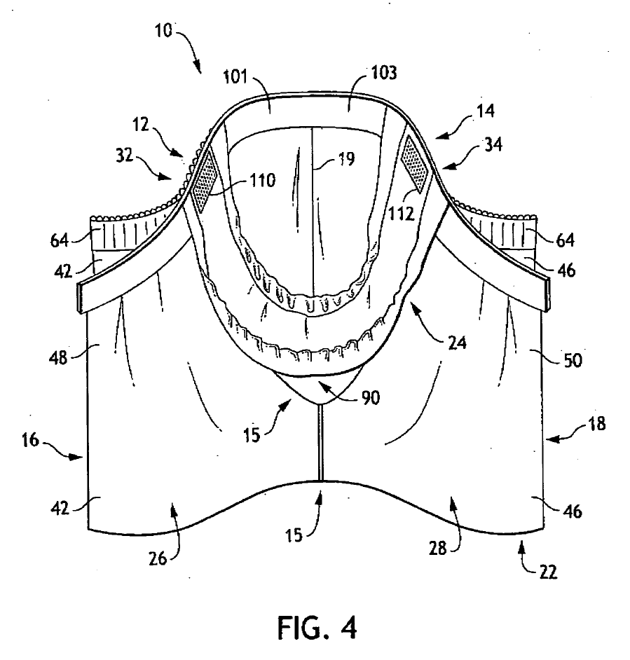

To gain a better understanding of the disposable absorbent garment 10, FIGS. 3 and 4 show a disposable absorbent garment 10 according to one aspect of the present invention, indicated in its entirety by the reference numeral 10. In this aspect, an absorbent member 24 may be permanently attached to a boxer short style garment shell 22 so that the entire absorbent garment 10 is disposable. In some aspects, the absorbent member may be removable, adjustable, or replaceable within the shell 22. The disposable absorbent garment 10 is configured to be worn on a wearer's lower torso and generally has a front waist region, indicated generally at 12, a back waist region, indicated generally at 14, and a crotch region, indicated generally at 15. In this embodiment, the front and back waist regions 12, 14 have respective side regions 16, 18 which are attached to each other along side seams 19.

With particular reference to FIGS. 3 and 4, the garment shell 22 comprises a front panel assembly, which is generally indicated at 26, having laterally opposite side margins 48 and a back panel assembly, which is generally indicated at 28 in FIG. 3, having laterally opposite side margins 50. The side margins 48, 50 of the front and back panel assemblies 26, 28 of the garment shell 22 are attached to each other to broadly define the side seams 19 of the absorbent garment 10, and to define the three-dimensional configuration of the garment shell during wear.

In its three-dimensional configuration as shown in FIG. 3, the garment shell 22 has a front waist region 32 which at least in part defines the front waist region 12 of the absorbent garment 10, a back waist region 34 which at least in part defines the back waist region 14 of the absorbent garment, and front and back waist ends, designated 56 and 58, respectively, which together generally define the waist opening 36 of the garment shell. In the illustrated embodiment, the garment shell 22 is configured to resemble a pair of shorts and thus further has a crotch region 38 extending longitudinally between and interconnecting the front waist region 32 and the back waist region 34 of the garment shell 22. The crotch region 38 of the garment shell 22 at least in part defines the crotch region 15 of the absorbent garment 10, and also in part defines leg openings 40 of the garment shell 22 (broadly referred to herein as outer leg openings of the absorbent garment). However, it is understood that the crotch region 38 of the garment shell 22 may be omitted (so that the crotch region 15 of the absorbent garment 10 is defined solely by the absorbent member 24 as described later herein), such as where the garment shell is intended to resemble a skort as defined herein (in which case only one leg opening 40 of the garment shell is provided to accommodate both legs of the wearer), without departing from the scope of this invention.

Referring to FIG. 3, the front panel assembly 26 of the garment shell 22 comprises a pair of panel members 42 which are in particular embodiments permanently attached to each other, along a central seam 44 extending longitudinally from the front waist region 32 to the crotch region 38 of the garment shell 22. The back panel assembly 28 comprises a pair of panel members 46 configured and permanently attached to each other in a manner similar to the panel members 42 of the front panel assembly 26 along a central seam (not shown) extending longitudinally from the back waist region 34 to the crotch region 38 of the garment shell 22. It is understood, however, that each of the front and back panel assemblies 26, 28 may be constructed of a single panel member (e.g., of unitary construction) without departing from the scope of this invention. Alternatively, the front and back panel members 42, 46 on one side of the garment shell 22 may be formed integrally at the crotch region 38 thereof so that no attachment of the panel members is necessary at the leg openings.

The panel members 42, 46 of the front and back panel assemblies 26, 28 of the garment shell 22 can be constructed of any suitable disposable material, and more suitably a material that provides a generally cloth-like texture. The garment shell is desirably intended to be disposable after a single use. As an example, the panel members 42, 46 may be constructed from natural and/or synthetic sources and may be constructed in any suitable manner including, but not limited, to non-absorbents such as spunbond-meltblown-spunbond film laminates, bonded carded web, spunlace, hydroentangled, and needle punched fabrics. The panel members 42, 46 are suitably liquid permeable, although it is understood that the panel members may be liquid impermeable without departing from the scope of this invention.

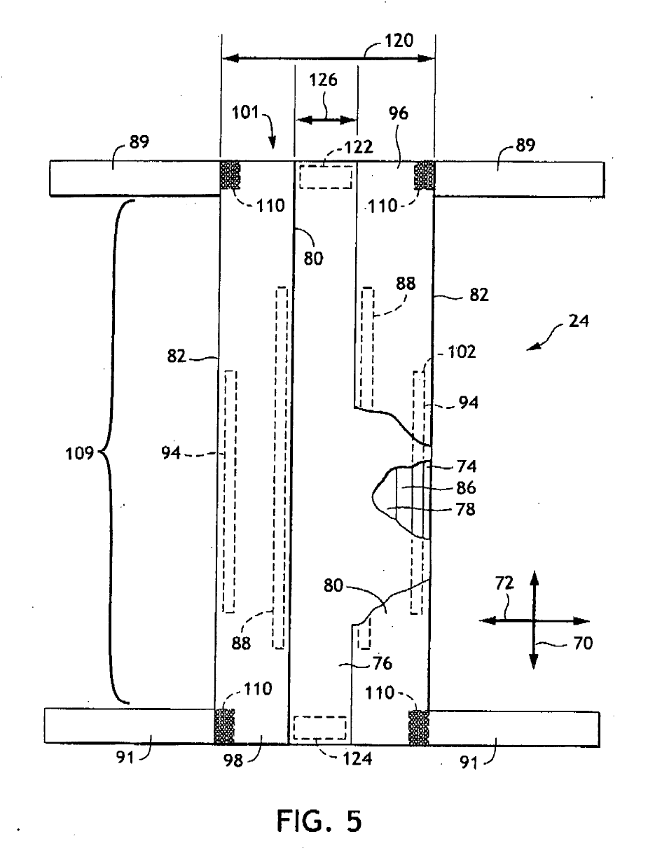

The absorbent member 24, as is illustrated in FIG. 5, is detached from the garment shell 22 and in a laid flat configuration. In some aspects, it is contemplated that the absorbent member 24 could be omitted from garment 10, leaving only the shell 22. In effect, this would create a non-absorbent garment for purposes of the present invention.

The absorbent member 24 is illustrated as being rectangular in shape, and has a longitudinal axis 70 and a transverse, or lateral axis 72. It is understood that the absorbent member 24 may be other than rectangular, such as hourglass-shaped, T-shaped, I-shaped or other suitable shape without departing from the scope of this invention. Further, the absorbent member 24 may be configured like a jock-strap, diaper, training pant, and the like.

The absorbent member 24 comprises an outer cover 74, a bodyside liner 76 in superposed relationship with the outer cover, an absorbent body 78 disposed between the outer cover and the bodyside liner, and a pair of laterally spaced containment flaps 80 configured to inhibit the transverse flow of body exudates on the liner to the side edges 82 of the absorbent member. The absorbent body 78 may contain fluff fiber, superabsorbent material, and/or other absorbent materials as desired. The absorbent member 24 may further include a front belt member 89 and a back belt member 91.

Each belt member 89, 91 of absorbent garment 10 is used to enhance the fit of the absorbent member 24 within absorbent garment 10 by forming an inner waist band 101, see FIGS. 3 and 4. More specifically, the belt members 89, 91, once attached to one another and to the absorbent member as described herein, are intended to provide tension along the waist ends 96, 98 of the absorbent member. It is further contemplated that the waist belts could be integrally connected to form a left belt member and a right belt member without departing from the intended scope of the invention. The belt members 89, 91 are suitably stretchable, and most desirably, suitably elastic.

The exterior cover 74 comprises a multi-layered laminate structure in which at least one of the layers is liquid impermeable. For instance, the exterior cover 74 can include a liquid permeable outer layer (not shown) and a liquid impermeable inner layer 86 which are suitably joined together by a laminate adhesive, ultrasonic bonds, pressure bonds, thermal bonds, or the like. The inner layer 86 of the exterior cover 74 can be both liquid and vapor impermeable, or it may be liquid impermeable and vapor permeable. Alternatively, the exterior cover 74 may comprise a single layer of liquid impermeable material.

The liquid permeable bodyside liner 76 is illustrated as overlying the exterior cover 74 and absorbent body 78, and may, but need not, have the same dimensions as the exterior cover 74. The bodyside liner 76 can be less hydrophilic than the absorbent body 78, to present a relatively dry surface to the wearer and to permit liquid to readily penetrate through the liner. Alternatively, the bodyside liner 76 can be more hydrophilic or can have essentially the same affinity for moisture as the absorbent body 78 to present a relatively wet surface to the wearer to increase the sensation of being wet. This wet sensation can be useful as a training aid. The hydrophilic/hydrophobic properties can be varied across the length, width and depth of the bodyside liner 76 and absorbent body 78 to achieve the desired wetness sensation or leakage performance.

The absorbent body 78 (FIG. 5) is positioned between the exterior cover 74 and the bodyside liner 76, which can be joined together by any suitable means.

Containment flaps 80 are located generally adjacent to the side edges 82 of the absorbent member 24, and can extend longitudinally along the entire length of the absorbent member 24 as shown in FIG. 5 or only partially along the length of the absorbent member. Flap elastic members 88 can be operatively joined with the containment flaps 80 in a suitable manner as is well known in the art. The elasticized containment flaps 80 can define a partially unattached distal edge (not shown), unattached to the liner 76, which assumes an upright configuration in at least the crotch region 90 of the absorbent member 24 during wear to form a seal (e.g., an elastic fit) against the wearer's body. It is understood, however, that the containment flaps 80 may be omitted without departing from the scope of this invention.

To enhance the fit of the absorbent garment 10 on the wearer and to further inhibit leakage of body exudates, the absorbent member may have leg elastic members 94 (FIG. 5), as are known to those skilled in the art. The leg elastic members 94 can be operatively joined to the exterior cover 74 and/or the bodyside liner 76 and extend longitudinally adjacent the opposite side edges 82 generally through the crotch region 90 of the absorbent member 24.

To further enhance the fit of absorbent garment 10, absorbent member 24 it is operatively attached to the garment shell at the front and back waist regions 32 and 34.

For additional detail regarding the construction of the pant, including the cover 74, the bodyside liner 76 and the absorbent body 78, reference may be made to the following patents/publications assigned to Kimberly-Clark Worldwide, Inc., incorporated herein by reference in a manner that is consistent herewith: U.S. Pat. Nos. 4,940,464; 4,938,757; and 6,613,033; and Patent Publication Nos. US20040127878; US20040102755; US20050085784; US20050256489; and US20060004341.

Various materials and methods for constructing disposable absorbent garments of the invention are known in the art. For example, an absorbent boxer may be manufactured using a process similar to that described in U.S. Pat. No. 7,192,500 to Kyle S. Allen, which is incorporated herein by reference in a manner that is consistent herewith. For example, the process generally defines a machine direction and a cross machine direction, and the process includes transporting a first web in the machine direction, the first web defining a first web first edge, a first web second edge opposite the first web first edge, and a first web interior within the first web first edge and the first web second edge. The process also includes transporting a second web in the machine direction, the second web defining a second web first edge, a second web second edge opposite the second web first edge, and a second web interior within the second web first edge and the second web second edge, where the second web is in a substantially superposed relationship to the first web. The process also includes removing portions of the first web and the second web, where the portions are substantially aligned to define an opening located within the first web interior and the second web interior. The process also includes attaching the first and second webs to form a crotch seam, and disposing an absorbent assembly on the first web proximate the opening while the first web first edge and the first web second edge are in a spaced relationship. In addition, the process includes directing a portion of the second web in the cross machine direction and overlapping at least a portion of the opening to define a second web overlapping portion, and attaching a portion of the absorbent assembly to one of the first and second webs.

In some aspects, the system and method of the present invention may also comprise a container, as defined above. In some particular aspects of the invention, the disposable absorbent garment 10 and the non-absorbent garment 510 are provided in separate containers. In other aspects of the invention, the disposable absorbent garment 10 and the non-absorbent garment 510 are provided in the same container. In addition, it is understood that more than one of disposable absorbent garment 10 and/or non-absorbent garment 510 may be present in a particular container.

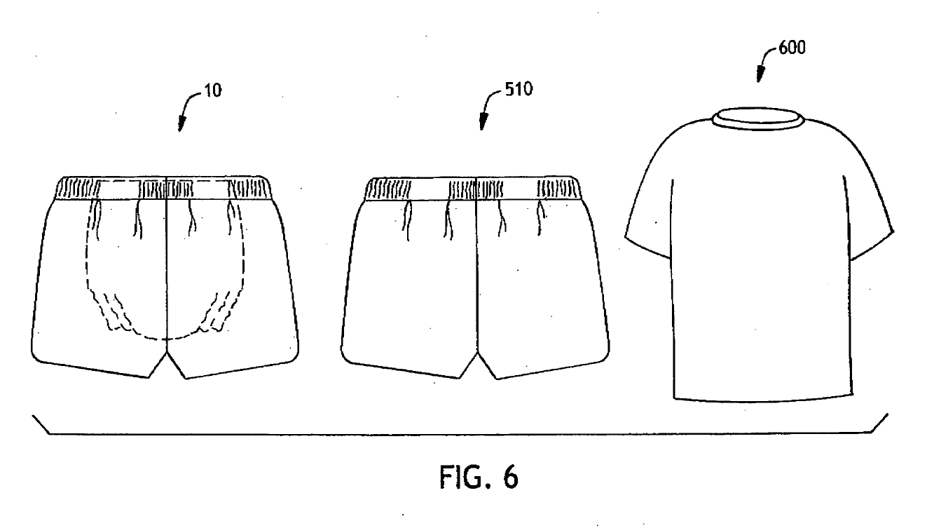

In some aspects, the system and method of the present invention further include a “secondary garment.” The secondary garment may be disposable or non-disposable, absorbent or non-absorbent, and woven or nonwoven. It is desirable that the secondary garment coordinates with the disposable absorbent garment 10 and/or the non-absorbent garment 510, but it need not substantially match. As used herein, the term “coordinate” refers to a garment that an average adult would utilize with, or have a common theme with, the disposable absorbent garment 10 or the non-absorbent garment 510. For example, an undershirt that has the same pattern as the disposable absorbent garment would coordinate with the disposable absorbent garment 10. FIG. 6 exhibits one exemplary embodiment of the present invention, wherein the system and method comprise a disposable absorbent garment 10, a non-absorbent garment 510, and a secondary garment 600 in the form of a shirt which coordinates with the other garments 10, 510 via a print pattern. In some aspects, the secondary garment 600 may be provided in the same container as the disposable absorbent garment 10 and/or the non-absorbent garment 510. In other aspects, the secondary garment 600 may be provided in a separate container.

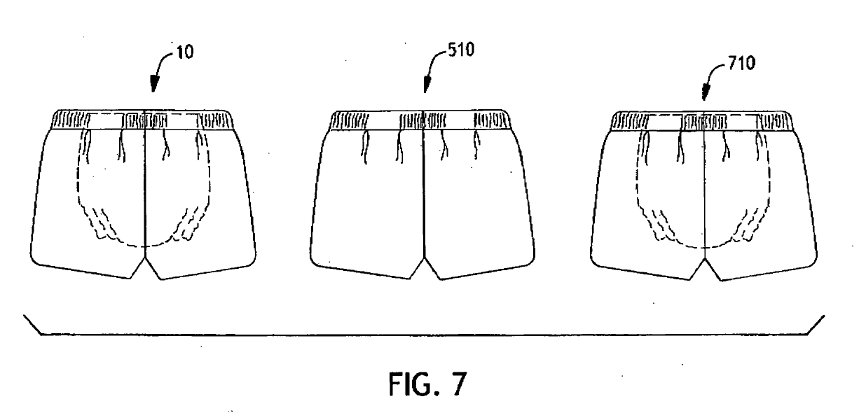

In some aspects the system and method of the present invention further include a “replacement garment.” Such replacement garments would include additional disposable absorbent garments that substantially match the non-absorbent garment 510 and/or the disposable absorbent garment 10. FIG. 7 exhibits one exemplary embodiment of the present invention, wherein the system and method comprise a disposable absorbent garment 10, a non-absorbent garment 510, and a replacement garment 710. In some aspects, the replacement garment 710 may be provided in the same container as the disposable absorbent garment 10 and/or the non-absorbent garment 510. In other aspects, the replacement garment 710 may be provided in a separate container. In still another aspect, a plurality of replacement garments 710 may be provided in the same container as or in a separate container from the disposable absorbent garment 16 and/or the non-absorbent garment 510.

In some aspects, a method of providing a system for enabling discretion comprises: providing a disposable absorbent garment 10; providing a non-absorbent garment 510; providing a container (not shown); bundling the disposable absorbent garment 10 and the non-absorbent garment 510 to form a bundled discretion product offering; and providing the bundled discretion product offering to a user; wherein the disposable absorbent garment 10 comprises a shell 22 and an absorbent member 24; wherein the shell 22 coordinates with the non-absorbent garment 510; and wherein the disposable absorbent garment 10 and the non-absorbent garment 510 are each a boxer. In other aspects, the method further comprises the step of providing a secondary garment 600, which optionally may be bundled with the disposable absorbent garment 10 and/or the non-absorbent garment 510. In yet another aspect, the method further comprises the step of providing at least one replacement garment 710, which optionally may be bundled with the disposable absorbent garment 10 and/or the non-absorbent garment 510.

It will be appreciated that details of the foregoing examples, given for purposes of illustration, are not to be construed as limiting the scope of this invention. Although only a few exemplary embodiments of this invention have been described in detail above, those skilled in the art will readily appreciate that many modifications are possible in the examples without materially departing from the novel teachings and advantages of this invention. For example, features described in relation to one example may be incorporated into any other example of the invention.

Accordingly, all such modifications are intended to be included within the scope of this invention, which is defined in the following claims and all equivalents thereto. Further, it is recognized that many embodiments may be conceived that do not achieve all of the advantages of some embodiments, particularly of the desirable embodiments, yet the absence of a particular advantage shall not be construed to necessarily mean that such an embodiment is outside the scope of the present invention. As various changes could be made in the above constructions without departing from the scope of the invention, it is intended that all matter contained in the above description shall be interpreted as illustrative and not in a limiting sense.

What is claimed is:

1. A hygiene discretion system comprising a disposable absorbent garment and a non-absorbent garment;
wherein the disposable absorbent garment comprises a shell and an absorbent member; and
wherein the shell substantially matches the non-absorbent garment.

2. The hygiene discretion system of claim 1 wherein the disposable absorbent garment and non-absorbent garment are each a boxer.

3. The hygiene discretion system of claim 1 wherein the shell substantially matches the non-absorbent garment via a garment theme.

4. The hygiene discretion system of claim 3 wherein the garment theme comprises a style.

5. The hygiene discretion system of claim 3 wherein the garment theme comprises a color.

6. The hygiene discretion system of claim 3 wherein the garment theme comprises a fabric texture.

7. The hygiene discretion system of claim 3 wherein the garment theme comprises a print pattern.

8. The hygiene discretion system of claim 3 wherein the garment theme is selected from style, color, fabric pattern, print pattern, or combinations thereof.

9. The hygiene discretion system of claim 1 wherein the shell has an opacity of at least 50%, as measured by the ISO Opacity Test.

10. The hygiene discretion system of claim 1 wherein the disposable absorbent garment and the non-absorbent garment comprise substantially the same looseness, as measured by the Garment Looseness Test.

11. The hygiene discretion system of claim 1 wherein the non-absorbent garment and the shell each cover at least 10% more surface area than the absorbent member of the disposable absorbent garment, as measured by the Surface Coverage Test.

12. The hygiene discretion system of claim 1 further comprising a container.

13. The hygiene discretion system of claim 12 wherein the disposable absorbent garment and the non-absorbent garment are provided in the same container.

14. The hygiene discretion system of claim 1 further comprising a secondary garment.

15. The hygiene discretion system of claim 14 wherein the secondary garment is a shirt.

16. The hygiene discretion system of claim 1 wherein the secondary garment coordinates with the non-absorbent garment.

17. The hygiene discretion system of claim 14 further comprising a container.

18. The hygiene discretion system of claim 17 wherein the disposable absorbent garment, the non-absorbent garment, and the secondary garment are provided in the same container.

19. The hygiene discretion system of claim 1 further comprising at least one replacement garment;
wherein the at least one replacement garment substantially matches the non-absorbent garment.

20. The hygiene discretion system of claim 19 wherein the at least one replacement garment is provided in a separate container from the disposable absorbent garment and non-absorbent garment.

21. A method of providing a system for enabling discretion comprising:
(A) providing a disposable absorbent garment;
(B) providing a non-absorbent garment;
(C) providing a container;
(D) bundling the disposable absorbent garment and the non-absorbent garment to form a bundled discretion product offering; and
(E) providing the bundled discretion product offering to a user;
wherein the disposable absorbent garment comprises a shell and an absorbent member;
wherein the shell coordinates with the non-absorbent garment; and
wherein the disposable absorbent garment and the non-absorbent garment are each a boxer.

22. The method of claim 21 further comprising the step of providing a secondary garment.

23. The method of claim 21 further comprising the step of providing at least one replacement garment.
